(19) United States
(12) Patent Application Publication
(10) Pub. No.: US 2025/0281387 A1
(43) Pub. Date: Sep. 11, 2025

(54) NATURAL COSMETIC PROCESS AND COMPOSITION

(71) Applicant: VooDoo Makeup, LLC, Katy, TX (US)

(72) Inventor: Aimée Carr, Katy, TX (US)

(21) Appl. No.: 18/350,746

(22) Filed: Jul. 12, 2023

Related U.S. Application Data
(60) Provisional application No. 63/388,213, filed on Jul. 11, 2022.

(51) Int. Cl. A61K 8/9794; A61K 8/37; A61K 8/92; A61K 8/9789; A61Q 19/00

(52) U.S. Cl. CPC A61K 8/9794; A61K 8/37; A61K 8/922; A61K 8/9789; A61Q 19/00; A61Q 19/005; A61K 2800/43

(57) ABSTRACT

A natural cosmetic process and composition is provided. The natural cosmetic composition comprises an amount of coconut solid, carnauba wax, pigment, MCT, meadowfoam, triglyceride, shea butter, emulsifiers and preservatives. The natural cosmetic composition that is safe and comfortable for users with allergies, is eco-friendly and sustainable in nature, is non-toxic, and does not come off from water, smearing, or rubbing.

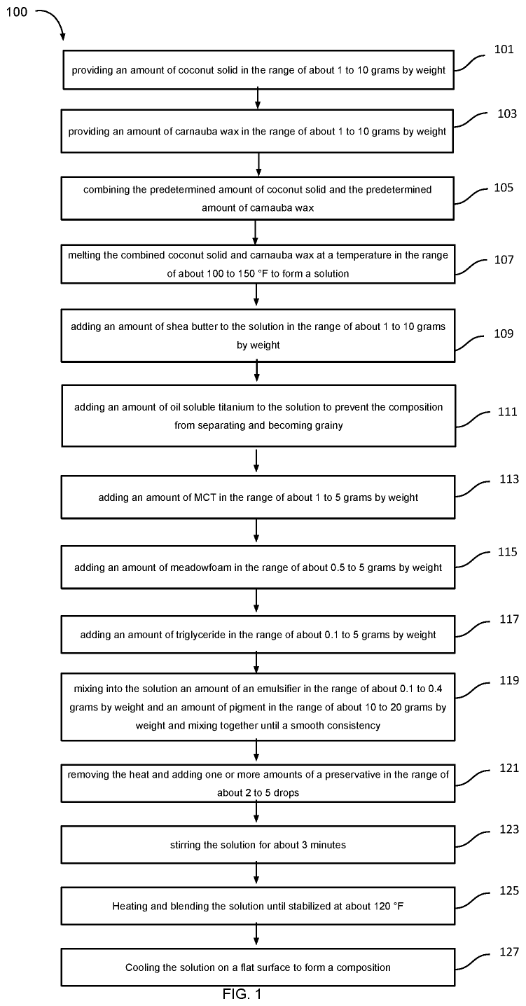

### CROSS-REFERENCE TO RELATED APPLICATION

This application claims the benefit of U.S. Provisional Patent Application No. 63/388,213 filed Jul. 11, 2022, which is incorporated by reference herein in its entirety.

### COPYRIGHT AND TRADE DRESS NOTICE

A portion of the disclosure of this patent document contains material that is subject to copyright or trade dress protection. This patent document may show and/or describe matter that is or may become trade dress of the owner. The copyright and trade dress owner has no objection to the facsimile reproduction by anyone of the patent disclosure, as it appears in the Patent and Trademark Office patent files or records, but otherwise reserves all copyright and trade dress rights whatsoever.

BACKGROUND

### 1. Field of the Invention

The present invention relates generally to cosmetics, and more specifically, to a natural cosmetic process and composition for safe and comfortable application.

### 2. Description of Related Art

This background information is intended to further educate the reader as to additional aspects of the prior art and may present examples of specific aspects of the prior art that is not to be construed as limiting the disclosure of the present application.

Application of cosmetic products is a common everyday activity for many individuals. A common problem associated with many cosmetic products is the effect it can have on a user with allergies. Conventional cosmetic products frequently contain a high percentage of chemicals or artificial ingredients and other harsh substances that can cause allergic reactions to many people. One dermatology study published in 2010 found that more than one-third of the 900 study participants experienced at least one allergic response to a cosmetic product. Symptoms may include simple rashes or more severe allergic responses. The onset of symptoms may occur immediately or after prolonged use.

Any part of the body may experience an allergic reaction, but the face, lips, eyes, ears, and neck areas are particularly susceptible to such reactions. Many common preservatives used in cosmetic products have been linked to skin allergies.

The concept of truly natural and organic cosmetics, whose composition should be primarily made of minerals, natural and organic plant extracts, products from microorganisms, or substances derived from these sources, is inconsistent with the use of chemical-based ingredients, despite the fact that they are regulated for safe use in cosmetics. These complex mixes' qualities, including those derived from oils and plant extracts, naturally aid in hydrating, nourishing, and protecting the skin. Additionally, the use of sustainable, organic, and biodegradable materials in natural and organic cosmetics reassures users about the reliability, provenance, and high caliber of these goods.

Natural and organic cosmetics not only help users protect and care for their skin naturally, but they also assist in managing overall health. Users feel more connected to nature and their senses are stimulated by their depiction of it through organic scents and subtle textures.

Products currently on the market either do not adequately treat the causes or symptoms of aging, the effects of external factors on the skin, or they irritate the skin. For instance, current skincare products might not address moisture loss, pigmentation issues, the appearance of fine lines or wrinkles, or loss of skin firmness.

Accordingly, although great advances have been made in the area of cosmetics, some deficiencies remain. There is a need for a natural cosmetic composition that is safe and comfortable to use with skin allergies.

### SUMMARY

The disclosure of the present application addresses the above stated deficiencies with cosmetics. The natural cosmetic process and composition of the present application is unique when compared with other known cosmetics because it provides a safe and comfortable application to users with allergies.

The disclosure of the present application describes a natural cosmetic composition comprising coconut solid, carnauba wax, pigment, MCT, meadowfoam, triglyceride, shea butter, emulsifiers and preservatives and method of preparing the composition.

An object of the present disclosure is to provide a natural cosmetic composition.

Another object of the present disclosure is to provide a natural cosmetic composition that is safe and comfortable for users with allergies.

Another object of the present disclosure is to provide a natural cosmetic composition that is eco-friendly and sustainable in nature.

Another object of the present disclosure is to provide a natural cosmetic composition that is applied topically.

Another object of the present disclosure is to provide a natural cosmetic composition that is non-toxic.

Another object of the present disclosure is to provide a natural cosmetic composition that does not come off from water, smearing, or rubbing.

Another object of the present disclosure is to provide a simple and economical preparation method for a natural cosmetic composition.

### DETAILED DESCRIPTION

Illustrative embodiments of the composition and method of the present application are provided herein. It should be appreciated that in the development of any actual embodiment, various implementation-specific decisions are required to achieve the developer's specific goals, such as compliance with system-related and business-related constraints, which vary from one implementation to another. Moreover, it should be appreciated that such a development effort might be complex and time-consuming but would nevertheless be a routine undertaking for those of ordinary skill in the art having the benefit of this disclosure.

The composition and method in accordance with the present application overcomes one or more of the above-discussed shortcomings commonly associated with cosmetics. Specifically, the natural cosmetic process and composition incorporates a means for allowing a user with allergies to safely and comfortably apply cosmetic products. These and other unique features of the composition and method are discussed below.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, elements, modules, units and/or components, but do not forbid the presence or addition of one or more other features, elements, components, and/or groups thereof. The particular order of steps disclosed in the method and process of the present disclosure is not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

The natural cosmetic composition of the present disclosure is free from the disadvantages of the prior art cosmetic compositions described hereinbefore, and is water resistant. Cosmetic compositions are common in everyday life and are generally used on a daily basis by many individuals. Particular needs are thus present for cosmetic compositions made from certain components for use in imparting various desired benefits to the skin and hair of a user.

The present disclosure relates to a natural cosmetic composition. The present disclosure also relates to a method of preparing the composition.

In an aspect, the present disclosure relates to a natural cosmetic composition comprising coconut solid, carnauba wax, pigment, MCT, meadowfoam, triglyceride, shea butter, emulsifier and preservatives.

In accordance with the present disclosure, coconut solid is present in the range of about 1 to 10 grams by weight. In an exemplary embodiment, the coconut solid is about 9.3 grams by weight.

In accordance with the present disclosure, carnauba wax is present in the range of about 1 to 10 grams by weight. In an exemplary embodiment, carnauba wax is about 3.9 grams by weight.

In accordance with the present disclosure, pigment is present in the range of about 10 to 20 grams by weight. In an exemplary embodiment, pigment is about 18.9 grams by weight.

In accordance with the present disclosure, MCT is present in the range of about 1 to 5 grams by weight. In an exemplary embodiment, MCT is about 2.4 grams by weight.

In accordance with the present disclosure, meadow foam is in the range of about 0.5 to 5 grams by weight. In an exemplary embodiment, meadow foam is about 1.5 grams by weight.

In accordance with the present disclosure, triglyceride is present in the range of about 0.1 to 5 grams by weight. In an exemplary embodiment, triglyceride is about 0.2 grams by weight.

In accordance with the present disclosure, shea butter is present in the range of about 1 to 10 grams by weight. In an exemplary embodiment, shea butter is about 6 grams by weight.

In accordance with the present disclosure, emulsifier is selected from the group consisting of sunflower lecithin, mustard, Diacetyl Tartaric Acid Esters of Monoglycerides (DATEM), PolyGlycerol Ester (PGE), Sorbitan Ester (SOE) and PG Ester (PGME). In an exemplary embodiment, the emulsifier is sunflower lecithin. Emulsions seek to improve the softness of the skin and prevent it from drying out during times of cold, dry weather.

In accordance with the present disclosure, the emulsifier is present in the range of about 0.1 g to 0.4 g. In an exemplary embodiment, the emulsifier is about 0.2 g.

In accordance with the present disclosure, preservatives are selected from a group consisting of rosemary, elderberry, neem oil, citric acid and grapeseed. In an exemplary embodiment, preservatives may include rosemary, elderberry, or a combination thereof.

In accordance with the present disclosure, preservative is present in the range of about 2 to 5 drops. In an exemplary embodiment, the preservative is about 3 drops.

In accordance with the present disclosure, composition may further comprise one or more of the ingredients listed in the list of cosmetic raw materials registered with the Food and Drug Administration and ingredients listed in ICID (International Cosmetic Ingredient Dictionary). For example, the cosmetic composition may include at least one of an organic solvent, a dissolving agent, a thickening agent, a gelling agent, a softening agent, an antioxidant, a suspending agent, a stabilizer, a fragrance agent, a surfactant, an emulsifier, a filler, a metal ion sequestrant, a preservative, a vitamin, a blocking agent, a wetting agent, dyes and pigments, hydrophilic or lipophilic active agents or auxiliary agents commonly used in the cosmetic field, such as any other natural ingredient conventionally used in cosmetics.

In accordance with the present disclosure, composition may contain vitamins selected from the group consisting of water-soluble and oil-soluble vitamins. The water-soluble vitamin can be formulated in cosmetics, preferably vitamin B1, vitamin B2, vitamin B6, pyridoxine, pyridoxine hydrochloride, vitamin B12, pantothenic acid, nicotinic acid, nicotinamide, folic acid, vitamin C, vitamin H, and the like, and more preferably may include vitamin C to help collagen synthesis.

In an embodiment, composition may further contain salts thereof (thiamine hydrochloride, sodium ascorbate salt, etc.) or derivatives thereof (ascorbic acid-2-sodium phosphate salt, ascorbic acid-2-magnesium phosphate salt and so on).

In accordance with the present disclosure, composition is formulated in the form selected from the group consisting of toners, emulsions, lotions, creams, pastes, gels, solutions, suspensions, oils, waxes, packs, powders, foundations, sprays, surfactant-containing cleansing, and the like, but it is not limited thereto. In an exemplary embodiment, the formulation is lotion. More specifically, it is prepared in the formulation of softening lotion, nourishing lotion, astringent lotion, nourishing cream, massage cream, milk lotion, powder, essence, eye cream, sun lotion, sun cream, makeup primer, makeup base, BB cream, powder foundation, emulsion foundation cleansing cream, cleansing foam, cleansing water, soap, pack, stick-form product, balm type product, spray or powder.

In an embodiment, when composition is a cream or gel formulation, it further includes animal oil, vegetable oil, wax, paraffin, starch, cellulose derivative, polyethylene glycol, silicone, bentonite, silica, talc or zinc oxide and the like, as the carrier component.

In accordance with the present disclosure, the composition may further include one or more carriers, excipients and/or additional ingredients suitable for topical administration.

In another aspect, the present disclosure relates to the method for preparation of the natural cosmetic composition. Initially, the coconut solid and carnauba wax are melted. In accordance with the present disclosure, the first predetermined amount of coconut solid and second predetermined amount of carnauba wax is melted at first predetermined temperature for a first predetermined time period to obtain a first solution.

In accordance with the present disclosure, a predetermined amount of coconut solid is in the range of about 1 to 10 grams by weight. In an exemplary embodiment, the amount is about 9.3 grams by weight.

In accordance with the present disclosure, a predetermined amount of carnauba wax is in the range of about 1 to 10 grams by weight. In an exemplary embodiment, the amount is about 3.9 grams by weight.

In accordance with the present disclosure, the predetermined amount of coconut solid and the predetermined amount of carnauba wax are combined and melted at a temperature in the range of about 100° F. to 150° F. to form a solution. In an exemplary embodiment, the temperature is about 120° F.

Next, shea butter is added to the solution. In accordance with the present disclosure, a predetermined amount of shea butter is added to the solution in the range of about 1 to 10 grams by weight. In an exemplary embodiment, shea butter is about 6grams by weight. The solution continues to melt at a temperature of about 120° F. Care is taken to keep the temperature on a low flame to avoid overheating and preventing the composition from forming.

Next, oil soluble titanium is added to the solution to avoid the graining and separation of phases of the composition and is melted.

In accordance with the present disclosure, a predetermined amount of MCT in the range of about 1 to 5 grams by weight is added to the solution. In an exemplary embodiment, the amount is about 2.4 grams by weight.

In accordance with the present disclosure, a predetermined amount of meadow foam in the range of about 0.5 to 5 grams by weight is added to the solution. In an exemplary embodiment, the amount is about 1.5 grams by weight.

In accordance with the present disclosure, a predetermined amount of triglyceride in the range of about 0.1 to 5 grams by weight is added to the solution. In an exemplary embodiment, the amount is about 0.2 grams by weight.

In the next step, pigment powder and emulsifier are added to the solution. The pigment is in the range of about 10 to 20 grams by weight. In an exemplary embodiment, pigment is about 18.9 grams by weight.

In accordance with the present disclosure, the pigment is poured on one side of the container holding the solution while the predetermined amount of emulsifier is poured on another side of the container. The composition is then mixed until smooth, maintaining a constant temperature.

In accordance with the present disclosure, emulsifier is selected from the group consisting of sunflower lecithin, mustard, Diacetyl Tartaric Acid Esters of Monoglycerides (DATEM), PolyGlycerol Ester (PGE), Sorbitan Ester (SOE) and PG Ester (PGME). In an exemplary embodiment, the emulsifier is sunflower lecithin. Emulsions seek to improve the softness of the skin and prevent it from drying out during times of cold, dry weather.

In accordance with the present disclosure, the emulsifier is present in the range of about 0.1 g to 0.4 g. In an exemplary embodiment, the emulsifier is about 0.2 g.

Next, heat is turned off and preservative is added to the solution and stirred for about 3 minutes to obtain a composition. In accordance with the present disclosure, a predetermined amount of preservative is added and heated again at a temperature of about 120° F.

In accordance with the present disclosure, preservative is selected from a group consisting of rosemary, elderberry, neem oil, citric acid and grapeseed. In an exemplary embodiment, preservative is rosemary. In another embodiment, preservative is elderberry.

In accordance with the present disclosure, the preservative amount is in the range of about 2-5 drops. In an exemplary embodiment, the amount is about 3 drops.

After stabilization and obtaining the desired consistency, the composition is transferred to an appropriate receptacle.

In accordance with the present disclosure, the mode of application is topical and is water resistant. In particular, in addition to being water resistant, the user generally expects to use such products, firstly, comfort of use and convenience of application and, secondly, good staying power over time. The properties of comfort of use and comfort of application are especially reflected by qualities such as softness, fluidity, shine and melting on the skin, and by a very fine and creamy texture. All these factors have been taken care of in the present invention.

In accordance with the present disclosure, application spots are selected from the group consisting of face, hand, any skin area and hair. In an exemplary embodiment, the application spot is face and hair. Skin creams seek to improve the softness of the skin and prevent it from drying out during times of cold, dry weather. Further, skin application products are commonly used for the prevention of acne and to aid in the healing of cuts or burns on the skin of the user.

In an embodiment, composition is taken and rubbed gently between hands to wake up the composition with the warmth of the skin and rubbing motion and then applied onto the skin.

In an embodiment, composition adjusts color and foundation to skin temperature.

In accordance with the embodiment of the present disclosure, compositions is capable of improving the general condition of the skin, provide healing properties, rejuvenating the skin, treating damage to the skin, improving the condition, or treating other conditions, disorders and diseases of the skin associated with changes in the components of the skin in humans.

In an embodiment, composition may be used according to a conventional method of use, and the number of times of use may vary depending on the skin condition or preference of the user.

The present disclosure described herein above has several technical advantages including, but not limited to, the realization of a natural cosmetic composition and method for preparation that: improving overall skin health; use of natural ingredients; water resistance; sustainable and on-toxic; comfort of use and application; free from mycotoxins, allergens, gluten, corn, and soy; and the process is simple and economical.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

The numerical values given for various physical parameters, dimensions, and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

The particular embodiments disclosed herein are illustrative only, as the embodiments may be modified and practiced in different but equivalent manners apparent to those skilled in the art having the benefit of the present disclosure. It is therefore evident that the particular embodiments disclosed herein may be altered or modified, and any such variations are considered to fall within the scope of the present application. Accordingly, the protection sought herein is as set forth in the description and the appended claims as well as any other variations and modifications falling within the scope thereof.

What is claimed is:

1. A natural cosmetic composition, comprising:
an amount of coconut solid in the range of about 1 to 10 grams by weight;
an amount of carnauba wax in the range of about 1 to 10 grams by weight;
an amount of pigment in the range of about 10 to 20 grams by weight;
an amount of MCT in the range of about 1 to 5 grams by weight;
an amount of meadow foam in the range of about 0.5 to 5 grams by weight;
an amount of triglyceride in the range of about 0.1 to 5 grams by weight;
an amount of shea butter in the range of about 1 to 10 grams by weight;
an amount of an emulsifier in the range of about 0.1 to 0.4 grams by weight; and
one or more amounts of a preservative in the range of about 2 to 5 drops.

2. The natural cosmetic composition of claim 1, wherein the amount of coconut solid is 9.3 grams.

3. The natural cosmetic composition of claim 1, wherein the amount of carnauba wax is 3.9 grams.

4. The natural cosmetic composition of claim 1, wherein the amount of pigment is 18.9 grams.

5. The natural cosmetic composition of claim 1, wherein the amount of MCT is 2.4 grams.

6. The natural cosmetic composition of claim 1, wherein the amount of meadow foam is 1.5 grams.

7. The natural cosmetic composition of claim 1, wherein the amount of triglyceride is 0.2 grams.

8. The natural cosmetic composition of claim 1, wherein the amount of shea butter is 6 grams.

9. The natural cosmetic composition of claim 1, wherein the amount of emulsifier is 0.2 grams.

10. The natural cosmetic composition of claim 1, wherein the one or more amounts of preservative is 3 drops.

11. A method of producing a natural cosmetic composition, the method comprising:
providing an amount of coconut solid in the range of about 1 to 10 grams by weight;
providing an amount of carnauba wax in the range of about 1 to 10 grams by weight;
combining the predetermined amount of coconut solid and the predetermined amount of carnauba wax;
melting the combined coconut solid and carnauba wax at a temperature in the range of about 100 to 150° F. to form a solution;
adding an amount of shea butter to the solution in the range of about 1 to 10 grams by weight;
adding an amount of oil soluble titanium to the solution to prevent the composition from separating and becoming grainy;
adding an amount of MCT in the range of about 1 to 5 grams by weight;
adding an amount of meadow foam in the range of about 0.5 to 5 grams by weight;
adding an amount of triglyceride in the range of about 0.1 to 5 grams by weight;
mixing into the solution an amount of an emulsifier in the range of about 0.1 to 0.4 grams by weight and an amount of pigment in the range of about 10 to 20 grams by weight and mixing together until a smooth consistency;
removing the heat and adding one or more amounts of a preservative in the range of about 2 to 5 drops;
stirring the solution for about 3 minutes;
Heating and blending the solution until stabilized at about 120° F.;
Cooling the solution on a flat surface to form a composition.

12. The method of producing a natural cosmetic composition of claim 11, wherein the amount of coconut solid is 9.3 grams.

13. The method of producing a natural cosmetic composition of claim 11, wherein the amount of carnauba wax is 3.9 grams.

14. The method of producing a natural cosmetic composition of claim 11, wherein the amount of pigment is 18.9 grams.

15. The method of producing a natural cosmetic composition of claim 11, wherein the amount of MCT is 2.4 grams.

16. The method of producing a natural cosmetic composition of claim 11, wherein the amount of meadow foam is 1.5 grams.

17. The method of producing a natural cosmetic composition of claim 11, wherein the amount of triglyceride is 0.2 grams.

18. The method of producing a natural cosmetic composition of claim 11, wherein the amount of shea butter is 6 grams.

19. The method of producing a natural cosmetic composition of claim 11, wherein the amount of emulsifier is 0.2 grams.

20. The method of producing a natural cosmetic composition of claim 11, wherein the one or more amounts of a preservative is 3 drops of rosemary and 3 drops of elderberry.
